(19) United States
(12) Patent Application Publication
(10) Pub. No.: US 2015/0265726 A1
(43) Pub. Date: Sep. 24, 2015

(54) ORGANIC NANOQUENCHER BASED ON CONJUGATED POLYMER AND PREPARATION METHOD THEREOF

(71) Applicant: University-Industry Foundation, Yonsei University, Seoul (KR)

(72) Inventors: Seungjoo HAAM, Seoul (KR); Yong-min HUH, Seoul (KR); Jaemoon YANG, Seoul (KR); Jihye CHOI, Incheon (KR); Eun Bi CHOI, Seoul (KR); Seo Ryung BAE, Seoul (KR); Doyeon BANG, Seoul (KR); Hyun-Ouk KIM, Seoul (KR); Taeksu LEE, Seoul (KR)

(21) Appl. No.: 14/614,545

(22) Filed: Feb. 5, 2015

(30) Foreign Application Priority Data
Mar. 20, 2014 (KR) 10-2014-0032971

(51) Int. Cl. A61K 49/00

(52) U.S. Cl. CPC A61K 49/0065; A61K 49/0054

(57) ABSTRACT

A pH responsive quencher based on a conjugated polymer and responding to an in vivo redox reaction and a preparation method thereof are provided. The pH responsive quencher can evaluate a metabolism environment according to a pH value in vivo with a characteristic that an absorption wavelength band sensitively changes depending on a pH condition.

### CROSS-REFERENCE TO RELATED APPLICATIONS

This application claims the benefit of priority of Korean Patent application No. 10-2014-0032971 filed Mar. 20, 2014, the contents of which are incorporated herein by reference in their entirety.

### BACKGROUND

1. Field of the Invention

The present invention relates to an organic nanoquencher based on a conjugated polymer and responding to an in vivo redox reaction and a preparation method thereof.

2. Background Art

In the life sciences, fluorescence-based techniques have been widely applied for defining various basic life phenomena from molecular biology to disease diagnosis. In particular, in addition to fluorescence intensity, fluorescence-based techniques use various experimental parameters including wavelength of excitation light, wavelength of fluorescence, fluorescence lifetime and fluorescence anisotropy, thereby making it possible to multiplex signals from a plurality of targets, and provide a resolution on a nanometer level and sensitivity on a single molecule level.

In recent years, conjugated polymers with repeating units comprising alternating double bond and single bond structures have drawn attention as high value-added materials such as organic electronic materials and sensor signal converting materials, which are high-tech functional materials. Such polymers are suitable to be applied to an active layer of organic solar cells, organic light emitting diodes, and organic transistors due to semiconductor electrical conductivity and absorption and emission in the visible light range caused by π-conjugated main chains and have also been researched and developed as organic electronic materials of the future due to their advantages such as flexibility of the materials, ease of structural transformation by way of organic synthesis, and mass producibility. Further, in recent years, research on application of the conjugated polymers to bio sensors and bio-imaging by imparting water solubility to the conjugated polymers and fusing the conjugated polymers into bio technology has been actively conducted.

Fluorescence resonance energy transfer (FRET), which is representative of the conventional methods for detecting a biomarker, is a method for detecting a difference in a fluorescence expression level caused by a difference in a distance between different fluorescent substances. In the fluorescence resonance energy transfer, two kinds of fluorescent substances are used, and thus a self-quenching phenomenon occurs frequently.

Further, metal quenchers which have been used in the conventional methods for detecting a biomarker cannot detect a change caused by an in vivo redox reaction.

### SUMMARY OF THE INVENTION

### Technical Problem

The present invention is directed to providing an organic nanoquencher based on a conjugated polymer capable of evaluating an in vivo redox metabolism environment with a characteristic that an absorption wavelength band sensitively responds to a redox reaction by combining an optical absorption property of an organic conjugated polymer and a quenching efficiency ratio between fluorescent substances and a preparation method thereof.

### Technical Solution

In order to solve the above problem, one aspect of the present invention provides a pH responsive quencher including: a first shell containing a conjugated polymer; a second shell covering the first shell; and a fluorescent substance dispersed on a surface of the second shell, wherein an inner core of the first shell has a hollow structure, and the fluorescent substance includes one or more of a first fluorescent substance which is color-developed in the range of pH 3 to pH 4.9, and a second fluorescent substance which is color-developed in the range of pH 5.1 to pH 7.

Further, in order to solve the above problem, another aspect of the present invention provides a preparation method of a pH responsive quencher including: a step of preparing a metal oxide in which a second shell containing one or more components selected from the group consisting of silica, titania, zirconia, and zeolite is formed; a step of forming a particle which has a hollow inner structure and in which a first shell containing a conjugated polymer and the second shell are formed in sequence by mixing the metal oxide, in which the second shell is formed, with a monomer of the conjugated polymer in an acid condition; and a step of attaching a fluorescent substance to the outside of the second shell.

Furthermore, in order to solve the above problem, still another aspect of the present invention provides a biomarker including a pH responsive quencher including: a first shell containing a conjugated polymer; a second shell covering the first shell; and a fluorescent substance dispersed on a surface of the second shell, wherein an inner core of the first shell has a hollow structure, and the fluorescent substance includes one or more of a first fluorescent substance which is color-developed in the range of pH 3 to pH 4.9, and a second fluorescent substance which is color-developed in the range of pH 5.1 to pH 7, and the biomarker satisfying the following condition (1) or (2):

(1) including a pH responsive quencher containing the first fluorescent substance together with a pH responsive quencher containing the second fluorescent substance; or

(2) including a pH responsive quencher containing the first fluorescent substance together with the second fluorescent substance.

### Effect of the Invention

A pH responsive quencher according to the present invention can evaluate a metabolism environment according to a pH value in vivo with a characteristic that an absorption wavelength band sensitively changes depending on a pH condition. Therefore, it becomes easy to detect an in vivo redox reaction often found in various metastatic cancers, and thus the pH responsive quencher can be used as a bioprobe which makes it possible to diagnose a malignant tumor early and determine a prognosis effectively.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 illustrates elements of a pH responsive quencher according to the present invention.

FIG. 2 is a schematic diagram illustrating a process for preparing the pH responsive quencher according to the present invention.

FIG. 3 is a graph showing different light absorption peak patterns of the pH responsive quencher depending on a change in a pH according to the present invention.

FIG. 4 is a graph showing fluorescence intensities of a first fluorescent substance and a second fluorescent substance depending on a pH according to a change in pH according to the present invention.

FIG. 5 is a schematic diagram illustrating a process in which a biomarker including the pH responsive quencher emits different fluorescences depending on a pH condition when moving in a cell.

### DETAILED DESCRIPTION OF THE INVENTION

The present invention can be modified and changed in various ways and can be embodied in various forms, and thus the present invention will now be described in detail with reference to the accompanying drawings, in which exemplary embodiments of the invention are illustrated.

It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Further, the elements illustrated in the accompanying drawings may be enlarged or reduced for convenience of explanation.

Hereinafter, the present invention will be described in detail with reference to the accompanying drawings, identical or corresponding elements will be assigned the same reference numerals throughout the figures, and any redundant description of identical or corresponding elements will be omitted.

Hereinafter, a pH responsive quencher according to the present invention will be described in detail.

In an exemplary embodiment of the present invention, there is provided a pH responsive quencher including: a first shell containing a conjugated polymer; a second shell covering the first shell; and a fluorescent substance dispersed on a surface of the second shell, wherein an inner core of the first shell has a hollow structure, and the fluorescent substance includes one or more of a first fluorescent substance which is color-developed in the range of pH 3 to pH 4.9, and a second fluorescent substance which is color-developed in the range of pH 5.1 to pH 7.

As used herein, the term “pH responsive quencher” refers to a particle which sensitively responds to a pH and marks a pH at a specific site depending on whether or not a color is developed by a fluorescent substance.

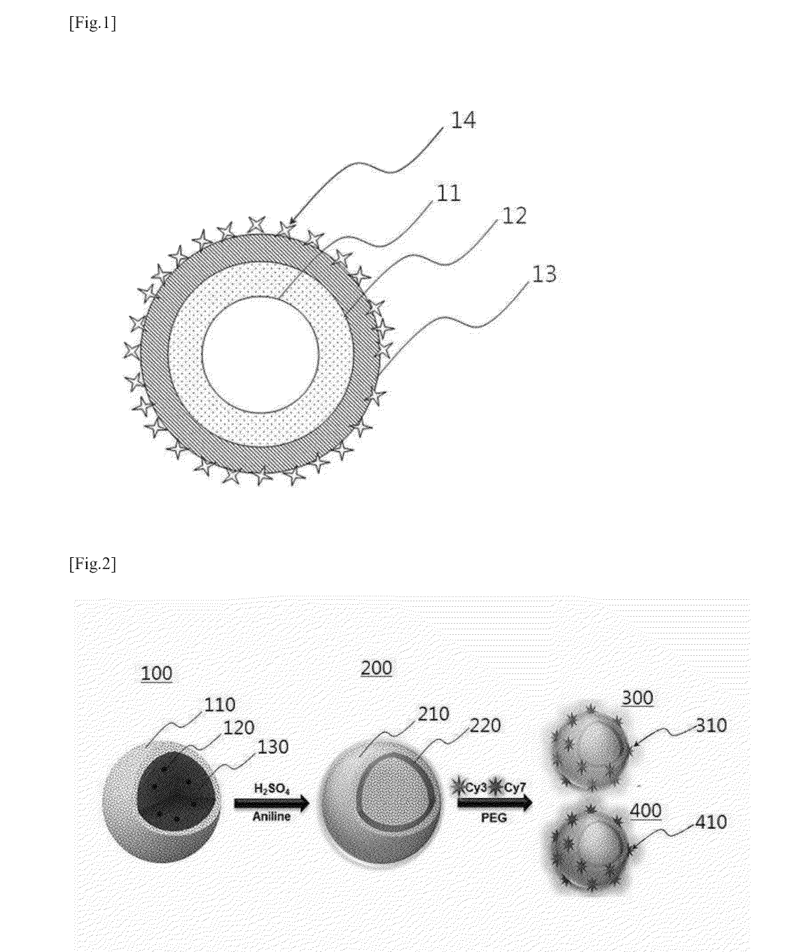

FIG. 1 illustrates a pH responsive quencher according to the present invention. Referring to FIG. 1, the pH responsive quencher may have a structure in which a first shell 12 having a hollow core structure 11 therein is covered by a second shell 13 and a fluorescent substance 14 is dispersed on a surface of the second shell 13.

According to the present invention, the first shell may contain a conjugated polymer. The conjugated polymer may be polyaniline. Further, the second shell may function as a support for the first shell. Polyaniline develops different colors in response to a change in a pH. However, if polyaniline develops a color in vivo, it is difficult to make an observation with a camera. Therefore, in the pH responsive quencher according to the present invention, the conjugated polymer shows a specific wavelength band depending on a pH in vivo and a quenching effect through the first fluorescent substance and second fluorescent substance dispersed on the surface of the second shell. Accordingly, it is possible to mark a pH in a body depending on whether or not colors are developed by the first fluorescent substance and the second fluorescent substance.

Therefore, in the pH responsive quencher according to the present invention, a specific wavelength band depending on a pH condition is combined with a fluorescent substance in which a quenching effect occurs, and thus, when the pH responsive quencher is introduced into the body, the pH responsive quencher sensitively responds to a pH in the body and marks a pH by way of color development of the first fluorescent substance and the second fluorescent substance in different colors, thereby evaluating a metabolism environment in vivo.

The fluorescent substance according to the present invention may be any fluorescent substance known to those skilled in the art. To be specific, the fluorescent substance may be a fluorescent dye which is released from a particle depending on a change in a pH so as to emit fluorescence. For example, the fluorescent substance may be Cy 3 or Cy 7 based on cyanine. Further, the fluorescent substance may be quantum dots. Furthermore, the fluorescent substance may be fluorescein and tetramethylrhodamine. Moreover, the fluorescent substance may be Texas red.

To be specific, according to the present invention, the fluorescent substances may satisfy the following formula 1.

pH₂−pH₁≦2 [Formula 1]

In formula 1,

pH₂ represents a pH range in which the second fluorescent substance develops a color, and

pH₁ represents a pH range in which the first fluorescent substance develops a color.

To be specific, the first fluorescent substance may develop a color in the range of pH 3 to pH 4.9 or pH 4.5 to pH 4.9. Further, the second fluorescent substance may develop a color in the range of pH 5.1 to pH 7 or pH 6.0 to pH 6.8.

In an exemplary embodiment of the present invention, the conjugated polymer contained in the first shell may include one or more selected from the group consisting of polyacetylene, polyaniline, polypyrrole, and polythiophene.

For example, the conjugated polymer may be formed of polyaniline. Polyaniline is easy to synthesize, environmentally safe, and easily doped. Further, polyaniline has high applicability in various fields due to its excellent workability, stability, economic efficiency, mechanical property, and conductivity.

In another exemplary embodiment of the present invention, the conjugated polymer may have a structure in which manganese ions or iron ions are coordinately bonded.

The conjugated polymer according to the present invention may be polyaniline, and the polyaniline has a structure in which manganese ions and iron ions are coordinately bonded. Thus, the conjugated polymer has a high solubility with respect to a solvent and also has an effect of regulating a band-gap level of the polyaniline by the coordinate bond.

In still another exemplary embodiment of the present invention, the second shell may coat an oxide containing manganese ions and iron ions and any material may be used without particular limitation as long as it is not changed in form and can support polyaniline when the polyaniline is formed. For example, the second shell may include one or more selected from the group consisting of silica, titania, zirconia, and zeolite. To be specific, the shell may be formed of silica.

In yet another exemplary embodiment of the present invention, the pH responsive quencher may have an average particle diameter of 0.1 nm to 100 μm. To be specific, the pH responsive quencher may have an average particle diameter of 5 nm to 10 μm. Further, the pH responsive quencher may have an average particle diameter of 10 nm to 200 nm.

Since the pH responsive quencher has an average particle diameter within the above-described range, it is easy to introduce the pH responsive quencher into the body and it becomes easier to mark a pH condition in the body.

Hereinafter, a preparation method of the pH responsive quencher according to the present invention will be described in detail.

In an exemplary embodiment of the present invention, there is provided a preparation method of a pH responsive quencher including: a step of preparing a metal oxide in which a second shell containing one or more components selected from the group consisting of silica, titania, zirconia, and zeolite is formed; a step of forming a particle which has a hollow inner structure and in which a first shell containing a conjugated polymer and the second shell are formed in sequence by mixing the metal oxide, in which the second shell is formed, with a monomer of the conjugated polymer in an acid condition; and a step of attaching a fluorescent substance to the outside of the second shell.

Further, in the preparation method of the pH responsive quencher according to the present invention, the fluorescent substance may include one or more of a first fluorescent substance which is color-developed in the range of pH 3 to pH 4.9, and a second fluorescent substance which is color-developed in the range of pH 5.1 to pH 7.

To be specific, the first fluorescent substance may develop a color in the range of pH 3 to pH 4.9 or pH 4.5 to pH 4.9. Further, the second fluorescent substance may develop a color in the range of pH 5.1 to pH 7 or pH 6.0 to pH 6.8.

Furthermore, in the preparation method of the pH responsive quencher according to the present invention, the metal oxide may be an oxide of one or more metals selected from manganese and iron.

Any material may be used as the oxide without particular limitation as long as it contains manganese and iron. Examples of an oxide which can be applied to the present invention may include MnO and Fe₃O₄.

MnO has excellent sensitivity, which make it possible to diagnose cancer in the body with high sensitivity. Further, Fe₃O₄ has excellent performance as a contrast medium for MRI. Therefore, if MnO and Fe₃O₄ are used in preparing a pH responsive quencher, it becomes easier to check whether or not a color is developed in the body by the pH responsive quencher.

According to the preparation method of the pH responsive quencher of the present invention, in the step of mixing the metal oxide, in which the second shell is formed, with the monomer of the conjugated polymer in an acid condition, the acid condition may be, for example, an aqueous solution in which one or more inorganic acids selected from the group consisting of sulfuric acid, hydrochloric acid, nitric acid, and phosphoric acid are mixed with water at a ratio of 1:5 to 15 (v/v). To be specific, the acid condition may be an aqueous solution in which sulfuric acid is mixed with water at a ratio of 1:10 (v/v).

Moreover, in the preparation method of the pH responsive quencher according to the present invention, the first shell may include the conjugated polymer in which manganese ions or iron ions are coordinately bonded.

The conjugated polymer according to the present invention may be polyaniline. The polyaniline has a structure in which manganese ions and iron ions are coordinately bonded to polyaniline. Thus, the conjugated polymer has a high solubility with respect to a solvent and also has an effect of regulating a band-gap level of the polyaniline by the coordinate bond.

To be specific, in the preparation method of the pH responsive quencher according to the present invention, by performing polymerization on a mixture of an aniline monomer dissolved in an aqueous hydracid solution and serving as a dopant and an oxidizing agent containing manganese ions and iron ions, it is possible to prepare polyaniline in a doped state in which manganese ions and iron ions derived from the oxidizing agent are coordinately bonded. Then, a pH responsive quencher can be prepared by bonding a fluorescent substance released from a particle and emitting fluorescence depending on a change in a pH to a surface of the polyaniline. Herein, a polyethyleneglycol (PEG)-based compound serving as a biocompatible polymer may be further added thereto.

The fluorescent substance used herein may be Cy 3 and/or Cy 7 based on cyanine. Further, the fluorescent substance may be quantum dots. Furthermore, the fluorescent substance may be fluorescein and/or tetramethylrhodamine. Moreover, the fluorescent substance may be Texas red.

Further, the present invention provides a biomarker including the pH responsive quencher.

Hereinafter, the biomarker according to the present invention will be described in detail.

To be specific, the biomarker includes: a first shell containing a conjugated polymer; a second shell covering the first shell; and a fluorescent substance dispersed on a surface of the second shell, wherein an inner core of the first shell has a hollow structure, and the fluorescent substance includes one or more of a first fluorescent substance which is color-developed in the range of pH 3 to pH 4.9 and a second fluorescent substance which is color-developed in the range of pH 5.1 to pH 7, and the biomarker satisfies the following condition (1) or (2):

(1) including a pH responsive quencher containing the first fluorescent substance together with a pH responsive quencher containing the second fluorescent substance; or

(2) including a pH responsive quencher containing the first fluorescent substance together with the second fluorescent substance.

To be specific, if the biomarker develops a color of the first fluorescent substance in vivo, it is possible to confirm that a pH value at an in vivo position of the biomarker is in the range of pH 3 to pH 4.9 or pH 4.5 to pH 5.0.

Further, if the biomarker develops a color of the second fluorescent substance in vivo, it is possible to confirm that a pH value at an in vivo position of the biomarker is in the range of pH 5.1 to pH 7 or pH 6.0 to pH 6.8.

Therefore, it is possible to check a pH condition in the body based on whether or not the two fluorescent substances are color-developed. Thus, it becomes easy to evaluate an in vivo redox metabolism environment.

In still another exemplary embodiment of the present invention, when the biomarker satisfies the condition (1), the pH responsive quencher containing the first fluorescent substance and the pH responsive quencher containing the second fluorescent substance may have a mixing weight ratio of 1:10 to 10:1.

To be specific, the mixing weight ratio may be 4:6 to 6:4, and since the first and second responsive quenchers have a mixing ratio within the above-described range, when the biomarker develops a color depending on a pH condition, it is possible to easily distinguish and check color development of the two fluorescent substances.

In still another exemplary embodiment of the present invention, the biomarker may identify a pH in the body from absorbance at a wavelength of 640 nm and absorbance at a wavelength of 840 nm.

To be specific, when the biomarker satisfies the following formula 2, a pH in the body is marked as 3 to 4.9 or 4.5 to 5.0, and when the biomarker satisfies the following formula 3, a pH in the body is marked as 5.1 to 7 or 6.0 to 6.8.

P₆₄₀−P₈₄₀≧0 [Formula 2]

P₆₄₀−P₈₄₀<0 [Formula 3]

In formulas 2 and 3, P₆₄₀ represents absorbance at a wavelength of 640 nm, and P₈₄₀ represents absorbance at a wavelength of 840 nm.

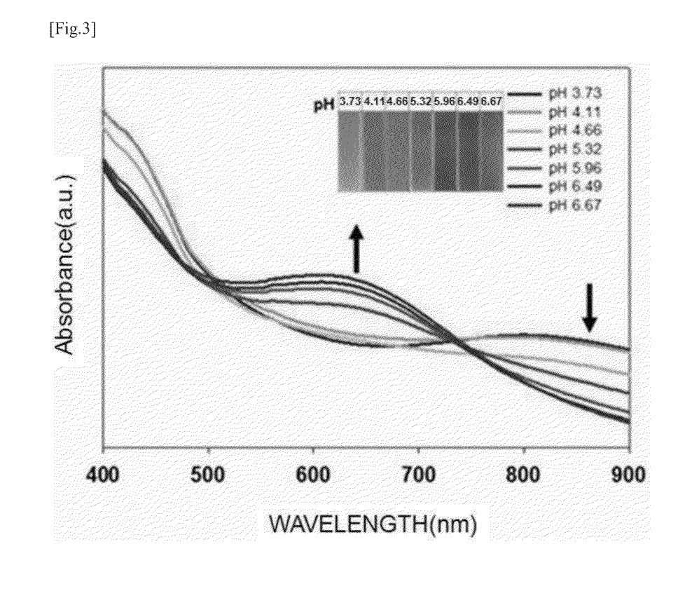

For example, FIG. 3 shows a change in absorbance of the pH responsive quencher at each wavelength. An arrow in FIG. 3 indicates a movement of an absorption peak according to an increase in a pH, and an inserted photo is a photo showing color development of a solution in which the pH responsive quencher is dispersed in water. Referring to FIG. 3, it can be seen that a light absorption peak becomes different even due to a sensitive change in a pH value of about 0.3.

According to the absorbance in a wavelength band of 640 nm of FIG. 3, as the pH value increases, the absorbance increases, and the absorption peak in the range of pH 5.32 to pH 6.67 is higher than the absorption peak in the range of pH 3.37 to pH 4.66. Further, in a wavelength band of 840 nm, as the pH value increases, the absorbance decreases, and the absorption peak in the range of pH 3.37 to pH 4.66 is higher than the absorption peak in the range of pH 5.32 to pH 6.67.

Therefore, if the condition of formula 2 is satisfied, light is absorbed from a fluorescent substance having a pH in the range of 5.1 to 7 or 6.0 to 6.8, and thus fluorescence intensity of the first pH responsive quencher containing the first fluorescent substance having a pH in the range of 3 to 4.9 or 4.5 to 5.0 becomes higher. Further, if the condition of formula 3 is satisfied, light is absorbed from a fluorescent substance having a pH in the range of 3 to 4.9 or 4.5 to 5.0, and thus fluorescence intensity of the second pH responsive quencher containing the second fluorescent substance having a pH in the range of 5.1 to 7 or 6.0 to 6.8 becomes higher.

Therefore, it can be seen that the biomarker including the pH responsive quencher according to the present invention sensitively responds to a change in a pH and shows various light absorption peaks depending on a wavelength range.

If the biomarker according to the present invention is introduced into the body, the two fluorescent substances show different fluorescence intensities depending on a pH condition in the body, and thus it is possible to check a pH condition in the body depending on whether or not colors are developed by the fluorescent substances.

For example, FIG. 5 shows an application example of a process in which a biomarker 10 including a pH responsive quencher emits different fluorescences depending on a pH condition when moving in a cell. Referring to FIG. 5, it can be seen that the biomarker 10 according to the present invention is sucked into a cell 40 and detects a section of an endolysosome depending on a pH.

Referring to FIG. 5, if the biomarker introduced into the cell moves to an endosome 20, fluorescence intensity of a second pH responsive quencher containing Cy 7 as a second fluorescent substance becomes strong. Therefore, it can be seen that the biomarker marks a region having a pH of 5.1 to 7.

If the biomarker 10 moves from the endosome 20 to a lysosome 30, fluorescence intensity of a first pH responsive quencher containing Cy 3 as a first fluorescent substance becomes strong. Therefore, it can be seen that the biomarker marks a region having a pH of 3 to 4.9.

As described above, it is possible to easily check a pH condition in the body with the biomarker including the pH responsive quencher that develops different colors depending on a pH.

The biomarker according to the present invention can detect a sensitive change in a pH. For example, with the measurement sensitivity to a change in a pH, the biomarker of the present invention can detect a change in a pH in a variation range of 1 or less, 0.7 or less, or 0.18 to 0.66.

### EXAMPLES

Hereinafter, the present invention will be described in more detail with reference to Examples and Experimental Examples.

However, the following Examples and Experimental Examples are provided only for illustration of the present invention, and do not limit the present invention.

### Example 1

### Preparation of First pH Responsive Quencher

100 μl of tetraethyl orthosilicate (TEOS) was added dropwise at a speed of 40 μl/hr to 0.01 g of MnO of 40 nm and 0.01 g of Fe₃O₄ of 12 nm to be used as oxidizing agents so as to perform silica coating thereon. Then, the coated MnO and Fe₃O₄ and 1 mL of an aniline monomer were added dropwise to a sulfuric acid aqueous solution. Herein, the aniline monomer, the sulfuric acid, and the water were mixed at a volume ratio of 1:2:20. The solution was vortexed for 30 minutes and centrifuged, thereby obtaining polyaniline nanoparticles in which purified MnO and Fe₃O₄ were coordinately bonded. Thereafter, Cy 3 was coated as a fluorescent substance on a surface of the polyaniline nanoparticle, thereby obtaining a first pH responsive quencher.

FIG. 2 is a schematic diagram illustrating a process for preparing a pH responsive quencher according to Example of the present invention.

Referring to FIG. 2, the process for preparing a pH responsive quencher will be described in detail. Firstly, Fe₃O₄ 120 and MnO 130 were coated with silica 110, thereby preparing a metal oxide 100 in which a second shell was formed. Polyaniline was formed by adding the metal oxide 100 in which a second shell was formed and an aniline monomer dropwise to a sulfuric acid (H₂SO₄) aqueous solution, and then, a particle 200 in which a first shell 220 as a shell containing the polyaniline and the second shell 210 as a silica shell covering the first shell 220 were formed in sequence was obtained. A surface of the particle 200 in which the first shell and the second shell were formed in sequence was coated with Cy 3 310 as a first fluorescent substance and Cy 7 410 as a second fluorescent substance, and herein, a polyethyleneglycol (PEG)-based compound serving as a biocompatible polymer may be further added thereto. Through the above-described process, a first pH responsive quencher 300 and a second pH responsive quencher 400 were prepared.

### Example 2

### Preparation of Second pH Responsive Quencher

A second pH responsive quencher was prepared and obtained in the same manner as Example 1 except that a surface of the prepared polyaniline particle was coated with a fluorescent substance Cy 7.

### Experimental Example 1

### Comparison of Light Absorption Peak Depending on pH Value

The following experiment was conducted in order to check absorbance of the pH responsive quencher of the present invention depending on a minimal change in a pH.

The first and second pH responsive quenchers prepared in Examples 1 and 2 were dispersed in water, and then a pH in the aqueous dispersion was measured and also absorbance at a wavelength of 400 nm to 900 nm was measured using and a UV spectrum. The absorbance was measured at a pH of the aqueous dispersion of 3.73, 4.11, 4.66, 5.32, 5.96, 6.49, and 6.67, and the result thereof was as shown in FIG. 3.

An arrow in FIG. 3 indicates a movement of an absorption peak according to an increase in a pH, and an inserted photo is a photo showing color development of a solution in which the pH responsive quencher is dispersed in water. Referring to FIG. 3, it can be seen that a light absorption peak becomes different even due to a sensitive change in a pH value of about 0.3.

According to the absorbance in a wavelength band of 640 nm of FIG. 3, the absorption peak in the range of pH 5.32 to pH 6.67 was higher than the absorption peak in the range of pH 3.37 to pH 4.66. Further, in a wavelength band of 840 nm, the absorption peak in the range of pH 3.37 to pH 4.66 was higher.

Therefore, it could be seen that the biomarker including the pH responsive quencher according to the present invention sensitively responds to a change in a pH and shows various light absorption peaks depending on a wavelength range.

### Experimental Example 2

### Comparison of Fluorescence Intensity Depending on pH Value

The following experiment was conducted in order to compare fluorescence intensity between the first pH responsive quencher and the second pH responsive quencher of the present invention depending on a pH.

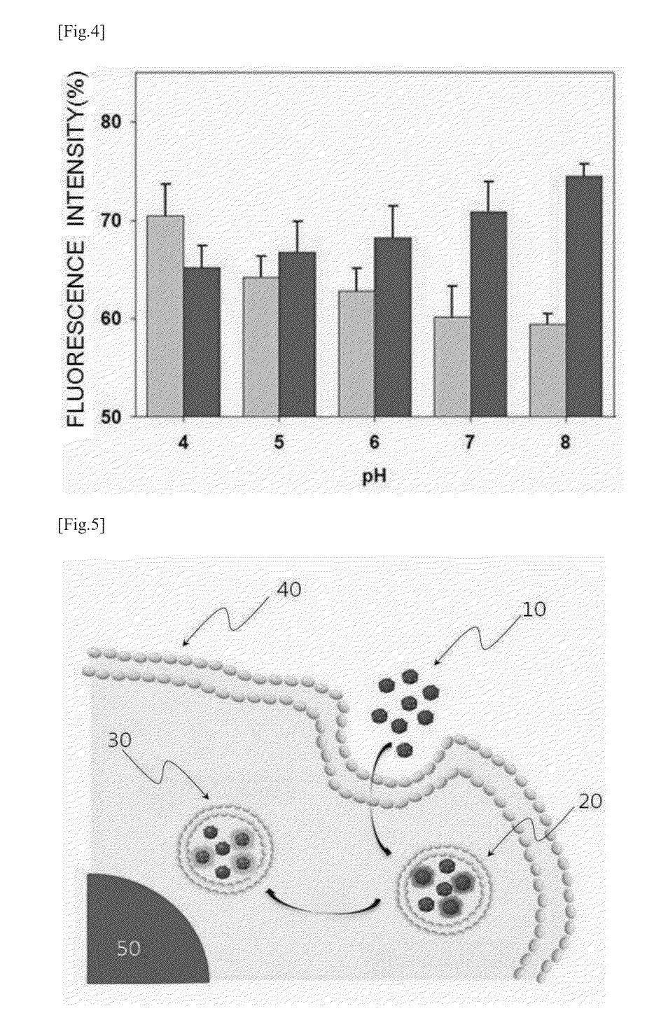

The first and second pH responsive quenchers prepared in Examples 1 and 2 were dispersed in water, and then a pH in the aqueous dispersion was measured and also fluorescence intensity depending on a pH condition was measured using a fluorescence spectrometer. The result thereof was as shown in FIG. 4.

Referring to FIG. 4, a yellow bar represents the fluorescence intensity of the first pH responsive quencher containing Cy 3 as the first fluorescent substance and a red bar represents the fluorescence intensity of the second pH responsive quencher containing Cy 7 as the second fluorescent substance. At a pH of about 4, the fluorescence intensity of the first pH responsive quencher was higher, and at a pH of 5 or more, the fluorescence intensity of the second pH responsive quencher was higher.

From the above-described experimental results, it could be seen that a pH responsive quencher can sensitively respond to a pH and regulate fluorescence intensity of a fluorescent substance.

Therefore, the pH responsive quencher according to the present invention causes color development of different fluorescent substances depending on a change in a pH, and thus it can be readily used in analyzing a pH condition in the body.

### EXPLANATION OF CODES

| | |
|---|---|
| 10: Biomarker | 20: Endosome |
| 30: Lysosome | 40: Cell |
| 50: Nucleus | |
| 11: Hollow structure | 12: First shell |
| 13: Second shell | 14: Fluorescent substance |

100: Metal oxide in which second shell is formed
110: Silica shell (first shell)
120: Fe₃O₄
130: MnO
200: Particle in which first shell and second shell are formed in sequence
210: Silica shell (second shell)
220: Polyaniline-containing shell (first shell)
300: First pH responsive quencher
310: First fluorescent substance (Cy 3)
400: Second pH responsive quencher
410: Second fluorescent substance (Cy 7)

What is claimed is:

1. A pH responsive quencher comprising:
a first shell containing a conjugated polymer;
a second shell covering the first shell; and
a fluorescent substance dispersed on a surface of the second shell,
wherein an inner core of the first shell has a hollow structure, and
the fluorescent substance includes one or more of a first fluorescent substance which is color-developed in the range of pH 3 to pH 4.9, and a second fluorescent substance which is color-developed in the range of pH 5.1 to pH 7.

2. The pH responsive quencher of claim 1, wherein the fluorescent substance satisfies the following formula 1:
pH₂−pH₁≦2 [Formula 1]
wherein, in formula 1,
pH₂ represents a pH range in which the second fluorescent substance develops a color, and
pH₁ represents a pH range in which the first fluorescent substance develops a color.

3. The pH responsive quencher of claim 1, wherein the conjugated polymer contained in the first shell includes one or more selected from the group consisting of polyacetylene, polyaniline, polypyrrole, and polythiophene.

4. The pH responsive quencher of claim 1, wherein the second shell includes one or more selected from the group consisting of silica, titania, zirconia, and zeolite.

5. The pH responsive quencher of claim 1, wherein the conjugated polymer has a structure in which manganese ions or iron ions are coordinately bonded.

6. The pH responsive quencher of claim 1, wherein the pH responsive quencher has an average particle diameter of 0.1 nm to 100 μm.

7. A preparation method of a pH responsive quencher comprising:
a step of preparing a metal oxide in which a second shell containing one or more components selected from the group consisting of silica, titania, zirconia, and zeolite is formed;
a step of forming a particle which has a hollow inner structure and in which a first shell containing a conjugated polymer and the second shell are formed in sequence by mixing the metal oxide, in which the second shell is formed, with a monomer of the conjugated polymer in an acid condition; and
a step of attaching a fluorescent substance to the outside of the second shell.

8. The preparation method of a pH responsive quencher of claim 7, wherein the fluorescent substance includes one or more of a first fluorescent substance which is color-developed in the range of pH 3 to pH 4.9, and a second fluorescent substance which is color-developed in the range of pH 5.1 to pH 7.

9. The preparation method of a pH responsive quencher of claim 7, wherein the metal oxide is an oxide of one or more metals selected from manganese and iron.

10. The preparation method of a pH responsive quencher of claim 7, wherein the first shell includes the conjugated polymer in which manganese ions or iron ions are coordinately bonded.

11. A biomarker comprising a pH responsive quencher including: a first shell containing a conjugated polymer; a second shell covering the first shell; and a fluorescent substance dispersed on a surface of the second shell, wherein an inner core of the first shell has a hollow structure, and
the fluorescent substance includes one or more of a first fluorescent substance which is color-developed in the range of pH 3 to pH 4.9, and a second fluorescent substance which is color-developed in the range of pH 5.1 to pH 7, and
the biomarker satisfying the following condition (1) or (2):
(1) including a pH responsive quencher containing the first fluorescent substance together with a pH responsive quencher containing the second fluorescent substance; or
(2) including a pH responsive quencher containing the first fluorescent substance together with the second fluorescent substance.

12. The biomarker of claim 11, wherein, when the biomarker satisfies the condition (1), the pH responsive quencher containing the first fluorescent substance and the pH responsive quencher containing the second fluorescent substance has a mixing weight ratio of 1:10 to 10:1.

13. The biomarker of claim 11, wherein the biomarker identifies a pH in the body from absorbance at a wavelength of 640 nm and absorbance at a wavelength of 840 nm.

14. The biomarker of claim 13, wherein, when the biomarker satisfies the following formula 2, a pH in the body is marked as 3 to 4.9, and when the biomarker satisfies the following formula 3, a pH in the body is marked as 5.1 to 7:
P₆₄₀−P₈₄₀≧0 [Formula 2]
P₆₄₀−P₈₄₀<0 [Formula 3]
wherein, in formulas 2 and 3, P₆₄₀ represents absorbance at a wavelength of 640 nm, and P₈₄₀ represents absorbance at a wavelength of 840 nm.
